(19) United States
(12) Patent Application Publication
(10) Pub. No.: US 2008/0140106 A1
(43) Pub. Date: Jun. 12, 2008

(54) Enhanced cuff sealing for endotracheal tubes

(75) Inventor: Kevin McGrath, Alpharetta, GA (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/638,020

(22) Filed: Dec. 12, 2006

(51) Int. Cl. A61M 29/00

(52) U.S. Cl. 606/191

(57) ABSTRACT

Gelling sealants that promote enhanced impermeability to medical devices is provided. The gelling sealants include various viscoelastic materials that exhibit a first viscosity upon introduction into a body cavity and a second viscosity after dwelling within the body cavity for a predetermined amount of time. The gelling sealants thicken and provide a bacterial barrier after introduction into a body cavity by adhering to the inside surfaces of the body cavity.

### BACKGROUND

Current endotracheal tubes and other sealing medical devices utilize an inflation cuff that presses against the sides of the trachea or other body cavity as a fluid barrier. The integrity of this cuff seal is critical, as there is a substantial amount of secretions that accumulate on top of the tubes during use that are harmful to the patient if allowed to enter the patient's lungs or pass beyond such other devices in other applications.

The inflation cuffs of traditional endotracheal tubes may slowly deflate over time and/or may be constructed of materials that allow gaps or spaces between the inflation cuff and the inside walls of the body cavity. For endotracheal tubes, these gaps allow for the passage or aspiration of secretions into the lungs. These secretions often contain bacteria that may cause ventilator acquired pneumonia which according to some studies accounts for 7-8% of all deaths in hospital Intensive Care Units.

Additionally, with endotracheal tubes, the inflation cuff can only be inflated with air or other substances to a certain predetermined level (usually 22+/−2 cm H2O), so that the inflation cuff does not cause damage to the trachea by exerting an excessive amount of force on the tracheal wall.

While much effort has been devoted to developing improved sealing cuffs, there is still a need for a way to enhance impermeability of a sealing cuff. More particularly, there is a need to enhance impermeability of a sealing cuff when inflation cuff sealing materials are constructed in a manner that allows gaps or spaces between the inflation cuff and inside walls of the body cavity and when the inflation cuff gradually deflates after insertion into the body cavity.

### SUMMARY OF INVENTION

The present invention provides for a gelling sealant for a medical device, the gelling sealant including a viscoelastic material. The gelling sealant containing the viscoelastic material has a first viscosity during introduction into the body cavity and a second higher viscosity after dwelling within the body cavity for a predetermined period of time. The medical device, which may include an inflation cuff, is adapted to occlude at least a portion of the body cavity. The gelling sealant containing the viscoelastic material provides enhanced impermeability to the portion the body cavity occluded by the medical device. That is, the gelling sealant containing the viscoelastic material may reduce the amount of undesirable secretions, liquids or fluids that are able to get past the portion of the body cavity occluded by the medical device during a predetermined period of time. In some embodiments, the gelling sealant containing the viscoelastic material may be able to substantially or completely eliminate undesirable secretions, liquids or fluids that are able to get past the portion of the body cavity occluded by the medical device during a predetermined period of time.

The gelling sealant first thickens and then adheres to the inner surfaces of the body cavity within a predetermined period of about 5 seconds to 5 hours. Generally speaking, the gelling sealant may thicken within about 5 minutes of less. The first viscosity of the gelling sealant ranges from about 1 to about 100 centipoise and the second viscosity of the gelling sealant ranges is at least about 1,000 centipoise, more desirably from at least about 100,000 centipoise to at least about 10,000,000 centipoise. The increase in viscosity from the first viscosity to the second viscosity may be prompted by crosslinking.

Desirably, the gelling sealant may be a hydrogel which may be formed from the interaction of divalent cationic and ionic polysaccharides. Suitable divalent cationic metal ions include Ca⁺² and Mg⁺². Suitable ionic polysaccharides include alginates, xanthan gums, natural gum, agarose, carrageenan, pectin, or amylopectin.

The gelling sealant may also include poly(oxyethylene)-poly(oxypropylene) block copolymers. The gelling sealant may include copolymers of poly(oxyethylene) with poly(styrene); poly(oxyethylene) with poly(caprolactone); or poly(oxyethylene) with poly(butadiene).

The gelling sealant may be formed from a mixture of poly(ethylene imines) having a molecular weight greater than 2000 daltons and poly(methacrylic acids) or poly(acrylic acids). The poly(ethylene imines) having a molecular weight greater than 2000 daltons may be dissolved in water to form a clear solution prior to mixing with poly(methacrylic acids) or poly(acrylic acids).

The gelling sealant may also include sucrose acetate isobutyrate solution or a solution that is 1 percent, by weight, agarose.

Another aspect of the invention addresses a system for reducing or eliminating leakage around medical devices and, more particularly, medical devices that include an inflation cuff. The medical device includes an inflation cuff and is adapted for introduction into a body cavity to occlude at least a portion of the body cavity. The viscoelastic material exhibits a first viscosity during introduction into a body cavity and a second, higher viscosity after dwelling within the body cavity for a predetermined period of time. Additionally, means for introducing the viscoelastic material into the body cavity adjacent the medical device is provided so that the material transitions to the second, higher viscosity thereby reducing or eliminating leakage around the inflation cuff.

Yet another aspect of the invention addresses a method for enhancing the impermeability of an inflation sealing cuff of a medical device. A medical device is provided that includes an inflation sealing cuff and is adapted for introduction into and occlusion of at least a portion of a body cavity. The medical device is inserted into the body cavity and the inflation sealing cuff is inflated. A viscoelastic material is delivered into the body cavity adjacent the inflation sealing cuff in a manner such that the viscoelastic material reduces or eliminates leakage around the inflation sealing cuff.

Another aspect of the invention addresses a method for providing a system of enhancing the impermeability of an inflation sealing cuff of a medical device, the method including the steps of: providing a medical device that includes an inflation sealing cuff and is adapted for introduction into and occlusion of at least a portion of a body cavity; providing a viscoelastic material that exhibits a first viscosity during introduction into the body cavity, and a second, higher viscosity after dwelling within the body cavity for a predetermined period of time, the body cavity being adjacent the inflation sealing cuff of the medical; and providing instructions that enable the viscoelastic material to be delivered to an area adjacent the inflation sealing cuff of the medical device within the body cavity such that the viscoelastic material provides enhanced impermeability to the inflation sealing cuff of the medical device.

Yet another aspect of the invention addresses kit for reducing or eliminating leakage around medical devices. The kit includes a medical device and a viscoelastic material. The medical device is adapted for introduction into a body cavity to occlude at least a portion of the body cavity. The viscoelastic material exhibits a first viscosity during introduction into the body cavity, and a second, higher viscosity after dwelling within the body cavity for a predetermined amount of time, the body cavity being adjacent the medical device adapted to occlude at least a portion of said body cavity.

### Definitions

As used herein, the terms “comprises”, “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof.

As used herein the term “viscosity” is the property of a fluid or semifluid that causes it to resist flowing. A liquid has a viscosity of one poise if a force of one dyne per square centimeter causes two parallel liquid on square centimeter in area and one centimeter apart to move past one another at a velocity of one centimeter per second.

One poise equals 100 centipoises (cp). Viscosity in centipoises divided by the liquid density at the same temperature gives kinematic viscosity in centistokes (cs). One hundred centistokes equal 1 stoke. To determine kinematic viscosity, the time is measured for an exact quantity of liquid to flow by gravity through a standard capillary.

Water is the primary viscosity standard with an accepted viscosity at 20 C of 0.01002 poise. Viscosity may be measured with conventional viscometers such as the appropriate model Brookfield Viscometer available from Brookfield Engineering Laboratories, Inc. 11 Commerce Boulevard Middleboro, Mass., USA, 02346

As used herein, the term “cohesion”, “cohesiveness”, and other derivatives from the root term “cohesive” is the property of a substance that allows it to stick, hold, or work together as a united whole. Additionally, it refers to the property of the gelling sealant within the body cavity that allows it form and maintain an integral mass within the body cavity despite changes in form or shape of the body cavity, the medical device, or any of the medical device attachments such as an inflation cuff.

As used herein the term “Ambient Conditions” is the temperature, pressure or relative humidity of air or other media in a designated area, particularly the area surrounding equipment. Generally speaking, for this disclosure, ambient temperature ranges from 68 to 72 Fahrenheit, pressure from 14 to 16 psia, and relative humidity in the range of 50 to 70 percent.

As used herein the term “adhesion”, “adhesiveness”, and other derivatives from the root word “adhesive” is the property of substance that allows it to bond to other substances by surface attachment. Adhesion is measured in terms of peel strength which is expressed in Newton/Centimeters (N/cm) and may be determined using conventional peel tests such as ASTM Standard Test Method D 1876 Standard Test Method for Peel Resistance of Adhesives or modified peel tests as described herein. With regard to the present application, “adhesive” refers to the ability of the gelling sealant to stick to the mucosal surface of the body cavity. Desirably, the gelling sealant will exhibit cohesive properties stronger than the adhesive properties.

As used herein the term “Biocompatible” is the nature of a substance that allows it to not irritate or damage the skin or living tissue while in contact with the skin or living tissue.

As used herein the term “gelling sealant” refers to a material or materials that includes at least one viscoelastic component or may itself exhibit viscoelastic properties when introduced into a body cavity adjacent a medical device and is adapted to thicken and increase in viscosity such that it reduces or eliminates leakage of liquids, secretions and/or fluids around the medical device.

As used herein, the term “viscoelastic material” refers to a material that exhibits both viscous and elastic properties. Examples of viscoelastic materials include polymers and asphalt. For purposes of the present invention, the elastic properties or characteristics of viscoelastic materials are less significant than the viscous properties. That is, the viscoelastic materials need only have a low level of elasticity so they are not brittle and have sufficient flexibility to avoid irritating the portion of a body cavity they may be used in.

### BRIEF DESCRIPTION OF DRAWINGS

FIG. 1. is an endotracheal tube having an inflation cuff and a gelling sealant.

### DETAILED DESCRIPTION

The invention disclosed herein is directed to a gelling material that reduces or eliminates leakage around a medical device, more particularly medical devices that may or may not have inflation cuffs. The medical device is inserted or placed in a body cavity and is adapted to occlude at least a portion of the body cavity. Non-limiting examples include endotracheal tubes and catheters such as bowel catheters, urinary catheters, and abdominal catheters. The reduction or elimination of leakage around the medical device, which may or may not have an inflation cuff, reduces or eliminates secretions or liquids from entering the lungs or other areas through aspiration. This occurs because, in use, there are a substantial amount of bodily secretions that accumulate on the side walls and on top of the inflation cuff of the medical device because the medical device is located in a body cavity. These secretions cannot be allowed to migrate into the lungs or other areas because of the presence of bacteria in the secretions. In this regard, the reduction or elimination of bacteria is important because it assists in the prevention of ventilator-acquired pneumonia, which according to some studies accounts for 7-8% of all deaths in hospital Intensive Care Units.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

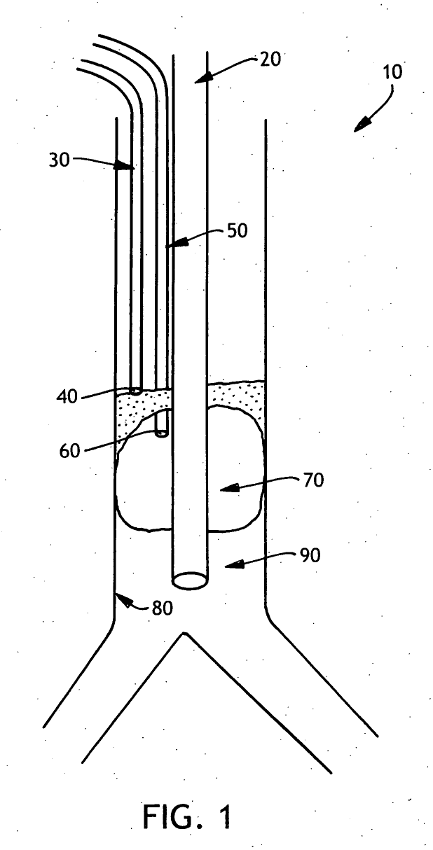

As illustrated in FIG. 1, a medical device 20 is inserted into a body cavity 90 to form a system 10 for reducing or eliminating leakage around the medical device. More particularly, an example of the type of medical device 20 that may be utilized is an endotracheal tube and examples of the body cavity 90 include an esophagus or trachea. It is contemplated, however, that any suitable medical device 20 may be used within any body cavity wherein secretions are capable of forming around the medical device and there is a desire to prevent or inhibit passage of the secretions past the device. Desirably, the medical device will have an inflation cuff 70 which will thereafter be inflated with air delivered through an air passageway 50 having an air passageway pore 60 in communication with the inside of the inflation cuff 70. However, the inflation cuff 70 may be inflated, with air, or any other substance effective for inflating the inflation cuff. Examples of other substances include saline solution and inert gases such as helium, neon, or molecular nitrogen. Additionally, the inflation cuff 70 may be inflated utilizing any method known in the art. Examples of inflation methods include inflation by means of a syringe, needle, cannula, catheter, or pressure applicator. An example of a medical device utilizing an inflation cuff is described in U.S. Pat. No. 6,526,977, to Gobel, issued Mar. 4, 2003, which is herein incorporated by reference in its entirety.

A gelling sealant including a viscoelastic material may then be introduced into a body cavity adjacent the inflation sealing cuff. The gelling sealant is biocompatible and may be introduced into the body cavity by any means known in the art. Examples of introduction methods include syringes, needles, cannulas, tubing, catheters, or pressure applicators. For illustrative purposes, FIG. 1 shows a gel passageway 30 used for delivery of the gelling sealant into a body cavity 90. The gelling sealant is first introduced into the gel passageway, it then flows through the gel passageway, and is then delivered into the body cavity 90 through a gel pore 40 or other opening. The inflation cuff, when inflated, substantially occludes the body cavity, however, the gelling sealant provides extra protection by flowing into any gaps remaining between the inflated cuff and the inner surfaces 80 of the body cavity in order to prevent secretions or fluids from entering the lungs or other areas. The gelling sealant exhibits an increase in viscosity when the gelling sealant is inside the body cavity and prevents secretions or fluids from entering the lung or other areas by reducing or eliminating leakage around the inflation sealing cuff.

In this regard, the gelling sealant comprises a viscoelastic material which exhibits a first viscosity during introduction into the body cavity. The temperature of the gelling sealing itself may range from about ambient temperature up to about 50 Celsius during introduction of the gelling sealant into the body cavity. Desirably, this first viscosity ranges from about 1 to about 100 centipoise. Desirably, the viscosity of the gelling sealant increases to at least about 1,000 centipoise, more desirably from about 100,000 centipoise up to about 10,000,000 centipoise after deposition in the body cavity. The temperature conditions inside the body cavity after deposition of the gelling sealant inside the body cavity ranges from about 32 Celsius to about 36 Celsius. An increase in viscosity to from about 100,000 centipoise up to about 10,000,000 centipoise may result in a near solidification or solidification of the gelling sealant.

The gelling sealant first thickens when deposited in the body cavity and then it may subsequently or simultaneously adhere to the inner surfaces of the body cavity within a period of about 5 seconds to 5 hours, more desirably from about 30 seconds to about 5 minutes. The thickening of the gelling sealant inside the body cavity is prompted by various mechanisms including temperature change and crosslinking. These mechanisms will be described in greater detail hereafter. Additionally, after thickening, the gelling sealant desirably adheres to the inner surfaces 80 of the body cavity in such a manner that the cohesion of the gelling sealant itself is stronger than the adhesion of the gelling sealant to the body this cavity. In this regard, the second viscosity gelling sealant may form a “plug” above the inflated cuff of the medical device, the plug being adapted to be removed from the body cavity without breaking apart or adapted to break apart in only a few large pieces. Advantageously, this helps prevent the gelling sealant from being aspirated into the lungs upon removal. Various biocompatible solvents or other liquids may be used assist in detachment of the gelling sealant from the inner surfaces of the body cavity and removal of the gelling sealant from the body cavity. These solvents or other liquids include water, alcohol, and other water-soluble biocompatible solvents or other liquids. Additionally, it is contemplated that solvents which are capable of changing the thermal conditions of the gelling sealing particles as well as the pH or strength of ionic bonds may be used to assist in detachment of the gelling sealant from the inner surfaces of the body cavity and removal of the gelling sealant from the body cavity.

Importantly, because the cohesion of the gelling sealant itself is stronger than the adhesion of the gelling sealant to the body cavity, the gelling sealant may provide a bacterial barrier whether the inflation cuff is inflated, deflated, or partially deflated. Additionally, the gelling sealant may be removed regardless of whether the inflation cuff is inflated, deflated, or partially deflated.

It is contemplated that a desirable characteristic of the gelling sealants herein is that their peel force, despite the fact the elastic modulus has a relatively low value (to ensure a high level of cohesiveness), is maintained at an appropriate value allowing them to exhibit appropriate adhesion performance on the surfaces of the body cavity. In order to ensure the required body cavity surface adhesion initially, and preferably over the entire period of medical device use, the gelling sealant may desirably have a peel strength on a body cavity surface of from 0.1 N/cm to 5 N/cm, more desirably from 0.3 N/cm to 3 N/cm, and yet even more desirably 0.3 N/cm to 2 N/cm as determined according to the test method described herein. It is believed that the peel force of the gelling sealant herein, as measured on a body cavity surface, may desirably be within those ranges.

Generally speaking, it is thought that the peel force to remove gelling sealant from a body cavity may be measured using a conventional peel test or modified peel test that utilizes a suitable tensile tester, for example an Instron Model 6021, equipped with a 10N load cell and an anvil rigid plate such as the Instron accessory model A50L2R-100. Gelling sealant samples may poured or otherwise deposited onto an artificial skin or forearm into strips of about width 25.4 mm and length between about 10 and 20 cm, allowed to thicken, and then rolled into place using a compression weight roller to prevent air entrapment between gelling sealant and skin. It is desirable that the weight roller be adapted to provide level compression to all parts of the samples. It is contemplated that to provide level compression to the samples, the weight roller may have diameter dimensions ranging from about 11 cm to about 16 cm, may have width dimensions between about 3 cm and about 5 cm, and may have a mass measurement of between about 3 and about 7 Kg. Additionally, the weight roller may be covered in rubber and have a rubber thickness ranging from about 0.3 to about 0.7 mm. The free end of the backing film may be attached to the upper clamp of the tensile tester and the arm placed below. The sample may be peeled from the skin at an angle of 90 degrees and a rate of 1000 mm/min. The average peel value obtained during peeling of the whole sample may be quoted as the peel value in N/cm. The average of triplicate measurements may be reported.

Gelling sealants may include a variety of materials that are desirably in the form of a liquid and may be a solution. Solutions desirable for use in accordance with the principles of the present disclosure include those that may be used to form coatings on tissue, and more particularly, thermal or viscous coatings. Mechanisms for forming coating include may form physical crosslinking, chemical crosslinking, or both.

Physical crosslinks may result from various processes including, but not limited to, complexation, hydrogen bonding, desolvation, Van der Waals interactions, ionic bonding, etc., and may be initiated by mixing two components that are physically separated until combined in situ, or as a consequence of a prevalent condition in the physiological environment, such as temperature, pH, ionic strength, etc. Physical crosslinking may be intramolecular or intermolecular or in some cases, both. For example, hydrogels can be formed by the ionic interaction of divalent cationic metal ions (such as Ca+2 and Mg+2) with ionic polysaccharides such as alginates, xanthan gums, natural gum, carrageenan, pectin, and amylopectin among others. These ionic polysaccharides are desirably found in aqueous solution in concentration of 5% by weight or higher. The formation of these hydrogels, formed from ionic polysaccharides, is not temperature dependent as the ionic crosslinking via divalent cations will proceed at room temperature. In this regard, the reaction rate and final viscosity are dependent upon the concentration of cations in the solution and not on the temperature. These crosslinks are easily reversed by exposure to species that chelate the crosslinking metal ions, for example, ethylene diamine tetraacetic acid (EDTA). Multifunctional cationic polymers, such as poly(L-lysine), poly(allylamine), poly(ethyleneimine), poly(guanidine), poly(vinyl amine), which contain a plurality of amine functionalities along the backbone, may be used to further induce ionic crosslinks

In some cases, block copolymers, graft copolymers, and/or homopolymers may be employed as gelling sealants. These gelling sealants are typically found in aqueous solution in concentration of 5% by weight or higher. For example, polyoxyalkylene block copolymers may be used in some embodiments of the present invention to form a thermo-gelling composition. The term “polyoxyalkylene block copolymers” refers to copolymers of alkylene oxides, such as ethylene oxide and propylene oxide, which form a gel when dispersed in water in a sufficient concentration. Some suitable polyoxyalkylene block copolymers include polyoxybutylene block copolymers and polyoxyethylene/polyoxypropylene block copolymers (“EO/PO” block copolymers), such as described in U.S. patent application Publication No. 2003/0204180 to Huang, et al., which is incorporated herein in its entirety by reference thereto for all purposes. For instance, exemplary polyoxyalkylene block copolymers include polyoxyethylene/polyoxypropylene block copolymers (EO/PO block copolymers) having the following general formula:

HO(CH₂CH₂O)ₓ(CH(CH₃)CH₂O)_{y}(CH₂CH₂O—)_{z}H

wherein,

x, y, and z are each integers in the range of about 10 to about 150.

The polyoxyethylene chain of such block copolymers typically constitutes at least about 60 wt. %, in some embodiments at least about 70 wt. % of the copolymer. Further, the copolymer typically has a total average molecular weight of at least about 2000, in some embodiments at least about 10,000, and in some embodiments, at least about 15,000. Suitable EO/PO polymers for use in the antimicrobial composition of the present invention are commercially available under the trade name PLURONIC® (e.g., F-127 L-122, L-92, L-81, and L-61) from BASF Corporation, Mount Olive, N.J.

Of course, any other thermogelling composition may also be used in the present invention. For example, other suitable thermogelling polymers may include homopolymers, such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N,n-diethylacrylamide); poly(N-isopropylmethacrylamide), poly(N-cyclopropylacrylamide), poly(N-ethylmethyacrylamide), poly(N-methyl-N-ethylacrylamide), poly(N-cyclopropylmethacrylamide), and poly(N-ethylacrylamide). Still other examples of suitable thermogelling polymers may include cellulose ether derivatives, such as hydroxypropyl cellulose, methyl cellulose, hydroxypropylmethyl cellulose, and ethylhydroxyethyl cellulose. Moreover thermogelling polymers may be made by preparing copolymers between (among) monomers, or by combining such homopolymers with other water-soluble polymers, such as acrylic monomers (e.g., acrylic or methacrylic acid, acrylate or methacrylate, acrylamide or methacrylamide, and derivatives thereof).

These thermogelling compositions exhibit thermoreversible behavior and exhibit weak physical crosslinks on warming. During introduction of thermoreversible solutions into a body cavity and upon contact with the body cavity tissues, target, viscosity is expected to increase from the formation of physical crosslinks. Similarly, pH responsive polymers that have a low viscosity at acidic or basic pH may be employed, and exhibit an increase in viscosity upon reaching neutral pH, for example, due to decreased solubility.

Several naturally existing polymers may be utilized as a hydrogel in the present disclosure. These naturally existing polymers function differently than the aforementioned thermogelling compositions because they exhibit a viscosity increase upon cooling. For example, gelatin, which is a hydrolyzed form of collagen, one of the most common physiologically occurring polymers, and which is typically found in 2 to 3 percent by weight aqueous solution, gels by forming physical crosslinks when cooled from an elevated temperature. In this regard, gelatin may be heated to a temperature up to about 50 Celsius so that it may desirably exhibit sufficient fluidity to be inserted into the body cavity. Upon cooling inside the body cavity, the gelatin becomes viscous. Similarly, a 1 percent, by weight, aqueous solution of agarose, upon cooling forms a flexible and resilient solid gel.

A change in solvent parameters may be used to induce a solidification from a low viscosity state to a high viscosity state. In this regard, U.S. Pat. No. 6,992,065, to Okumu, issued Jan. 31, 2006 describes an additional system that does not require a temperature change. In Okumu, sucrose acetate isobutyrate is used as an injectable drug delivery system—in ethanol it is a highly fluid solution, but once in contact with human tissues a rapid replacement of the ethanol with aqueous solutions triggers a solidification.

### EXAMPLES

### Example 1

The following example describes the barrier properties of the gelling sealant. An adult Kimberly-Clark Microcuff endotracheal tube manufactured by Microcuff GmbH of Weinheim, Germany, and a Mallinckrodt Hi-Lo endotracheal tube manufactured by Mallinckrodt, Inc. of St. Louis, Mo. were each placed into a 50 mL graduated cylinder of inner diameter 21-22 mm (to simulate a trachea). The cuffs were inflated with air to 22+/−2 cm H₂O (the manufacturer's specifications). In one set of studies 5 mL of a 1 wt % aqueous agarose solution available from Sigma Chemical Company of St. Louis was warmed to 50 degrees Celsius so that it flowed freely and was readily pourable and layered on top of each tube and allowed to solidify over a period of five minutes at ambient temperature. A control set of tubes including an adult Kimberly-Clark Microcuff endotracheal tube and a Mallinckrodt Hi-Lo endotracheal tube were inflated with air to 22+/−2 cm H2O (the manufacturer's specifications) at ambient temperature, but no gel plug was applied onto the top of the tubes. Each set of tubes was challenged with 10 mL of deionized water containing a FD and C red food dye #1 for improved visualization. After less than 10 minutes, the Mallinckrodt tube had leaked several mL of solution, while the Microcuff tube had fluid migration slightly down the cuff, but no leakage. After 24 hrs at ambient temperature, the Mallinckrodt tube had leaked completely, and the Microcuff tube had a very small amount of leakage (under 200 uL). Both the adult K-C Microcuff endotracheal tube and the Mallinckrodt Hi-Lo endotracheal tube containing the agarose gel plugs showed no leakage, even after 24 hrs—a dramatic improvement over both the control Mallinckrodt tube and the control Microcuff tube.

The elimination of leakage in both the Mallinckrodt and Microcuff tubes containing agarose gel plugs is particularly important because secretions containing bacteria may build up upstream or behind medical devices inserted into body cavities. In the case of endotracheal tubes it is important to keep these secretions from being aspirated into the lungs to help avoid aspirating secretion that might cause ventilator-acquired pneumonia. In this regard, approximately 8-28 percent of critical care patients on mechanical ventilators may develop VAP. Additionally, some studies suggest that VAP patients have a mortality rate of 20-33 percent, and VAP increases patient time in the ICU by 4-6 days and adds an average cost of $20,000-40,000 per incident.

### Example 2

An adult Kimberly-Clark Microcuff endotracheal tube and a Mallinckrodt Hi-Lo endotracheal tube were each placed into a 50 mL graduated cylinder of inner diameter 21-22 mm (to simulate a trachea) at ambient temperature. The cuffs were inflated with air to 22+/−2 cm H2O (the manufacturer's specifications). In one set of studies 5 mL of a 1 wt % aqueous agarose solution, warmed to 50 Celsius so that it flowed freely and was readily pourable, was layered on top of each tube and allowed to solidify over a period of five minutes at ambient temperature. A control set of tubes including an adult Kimberly-Clark Microcuff endotracheal tube and a Mallinckrodt Hi-Lo endotracheal tube were inflated with air to 22+/−2 cm H2O (the manufacturer's specifications), but no gel plug was applied onto the top of the tubes. In each case, the inflation cuff of the both the Mallinckrodt and Microcuff tubes having the 1% aqueous agarose solution as well as the control tubes were completely deflated using a syringe to withdraw air from the cuff. The control samples all leaked immediately, but the tubes with the secondary gel cuff showed no leakage, despite the complete deflation of the cuff.

The fact that neither the K-C Microcuff endotracheal tube nor the Mallinckrodt Hi-Lo endotracheal tube is significant because gradual leakage of inflation cuffs occurs when an endotracheal tube is inside a body cavity. When a gel is not used, the gradual deflation of the cuff will form creases and pockets between the inflation cuff and the insides of the body cavity allowing bacteria to pass into the lungs.

However, because the gel plugs utilized in both the K-C Microcuff endotracheal tube and the Mallinckrodt Hi-Lo endotracheal tube exhibit cohesion stronger than the adhesion of the gelling sealant to the body this cavity, the particles of the gelling sealant remain in continuity with each other even as the shape of the inflation cuff changes. Thus, the gelling sealant continues to act as a secretion barrier even as the inflation cuff deflates.

What is claimed is:

1. A gelling sealant for a medical device, said gelling sealant comprising:
a viscoelastic material exhibiting a first viscosity during introduction into a body cavity; and
a second viscosity after dwelling within the body cavity for a predetermined amount of time, said body cavity being adjacent a medical device adapted to occlude at least a portion of said body cavity;
said viscoelastic material providing enhanced impermeability to the portion of said body cavity occluded by the medical device.

2. The gelling sealant of claim 1, wherein said medical device includes an inflation cuff.

3. The gelling sealant of claim 1, wherein said gelling sealant thickens and then adheres to the inner surfaces of said body cavity after dwelling within the body cavity for a predetermined amount of time.

4. The gelling sealant of claim 3, wherein the predetermined amount of time is from about 5 seconds to about 5 hours.

5. The gelling sealant of claim 4, wherein the predetermined period of time is from about 30 seconds to about 5 minutes.

6. The gelling sealant of claim 1, wherein said first viscosity of said gelling sealant ranges from about 1 to about 100 centipoise.

7. The gelling sealant of claim 1, wherein said second viscosity of said gelling sealant is at least about 1,000 centipoise.

8. The gelling sealant of claim 7, wherein said second viscosity of said gelling sealant ranges from about 100,000 to about 10,000,000 centipoise.

9. The gelling sealant of claim 1, wherein the gelling sealant is formed from crosslinking.

10. The gelling sealant of claim 9, wherein said crosslinking is reversible.

11. The gelling sealant of claim 1, wherein the gelling sealant comprises a hydrogel.

12. The gelling sealant of claim 11, wherein said hydrogel is formed from the interaction of divalent cationic metal ions and ionic polysaccharides.

13. The gelling sealant of claim 12, wherein said divalent cationic metal ions are Ca⁺² or Mg⁺².

14. The gelling sealant of claim 12, wherein said ionic polysaccharides are alginates, xanthan gums, natural gum, carrageenan, pectin, or amylopectin.

15. The gelling sealant of claim 1, wherein said gelling sealant comprises poly(oxyethylene)-poly(oxypropylene) block copolymers.

16. The gelling sealant of claim 1, wherein said gelling sealant comprises copolymers, and wherein said copolymers are copolymers of poly(oxyethylene) with poly(styrene); poly(oxyethylene) with poly(caprolactone); or poly(oxyethylene) with poly(butadiene).

17. The gelling sealant of claim 1, wherein said gelling sealant is formed from a mixture of poly(ethylene imines) having a molecular weight greater than 2000 daltons and poly(methacrylic acids) or poly(acrylic acids).

18. The gelling sealant of claim 17, wherein said poly(ethylene imines) having a molecular weight greater than 2000 daltons is dissolved in water to form a clear solution prior to mixing with poly(methacrylic acids) or poly(acrylic acids).

19. The gelling sealant of claim 1, wherein said gelling sealant comprises a solution that is 1 percent, by weight, agarose.

20. The gelling sealant of claim 1, wherein said gelling sealant comprises sucrose acetate isobutyrate.

21. A system for reducing or eliminating leakage around medical devices that include an inflation cuff, said system comprising:
a medical device that includes an inflation cuff and is adapted for introduction into a body cavity to occlude at least a portion of said body cavity;
a viscoelastic material exhibiting:
a first viscosity during introduction into a body cavity; and
a second viscosity after dwelling within the body cavity for a predetermined amount of time, said body cavity being adjacent said medical device adapted to occlude at least a portion of said body cavity; and
means for introducing said viscoelastic material into said body cavity adjacent said medical device so that the material transitions to the second, higher viscosity thereby reducing or eliminating leakage around the inflation cuff.

22. The system of claim 21, wherein said means for introducing said viscoelastic material into said body cavity includes a tube adapted to receive a gelling material and introduce said gelling material into said body cavity adjacent said inflation cuff.

23. The system of claim 21, wherein said viscoelastic material thickens and then adheres to the inner surfaces of the body cavity after dwelling within the body cavity for a predetermined amount of time.

24. A method for enhancing the impermeability of an inflation sealing cuff of a medical device comprising:
providing a medical device that includes an inflation sealing cuff and is adapted for introduction into and occlusion of at least a portion of a body cavity;
inserting said medical device into said body cavity;
inflating said inflation sealing cuff; and
delivering a viscoelastic material into said body cavity adjacent said inflation sealing cuff in a manner such that said viscoelastic material reduces or eliminates leakage around the inflation sealing cuff.

25. The method of claim 24, wherein said viscoelastic material thickens and then adheres to the inner surfaces of the body cavity after dwelling within the body cavity for a predetermined amount of time.

26. A method for providing a system of enhancing the impermeability of an inflation sealing cuff of a medical device comprising:
providing a medical device that includes an inflation sealing cuff and is adapted for introduction into and occlusion of at least a portion of a body cavity;
providing a viscoelastic material exhibiting:
a first viscosity during introduction into a body cavity; and
a second, higher viscosity after dwelling within the body cavity for a predetermined amount of time, said body cavity being adjacent said inflation sealing cuff of said medical device adapted to occlude at least a portion of said body cavity; and
providing instructions to deliver the viscoelastic material to an area adjacent the inflation sealing cuff of the medical device within the body cavity such that the viscoelastic material provides enhanced impermeability to the inflation sealing cuff of the medical device.

27. The method of claim 26, wherein a delivery means is provided to deliver the viscoelastic material into the body cavity.

28. The method of claim 27, wherein the delivery means is a syringe, needle, cannula, catheter, tubing, or pressure applicator.

29. A kit for reducing or eliminating leakage around medical devices, said kit comprising:
a medical device that is adapted for introduction into a body cavity to occlude at least a portion of said body cavity;
a viscoelastic material exhibiting:
a first viscosity during introduction into a body cavity; and
a second viscosity after dwelling within the body cavity for a predetermined amount of time, said body cavity being adjacent said medical device adapted to occlude at least a portion of said body cavity.

30. The kit of claim 29, wherein the medical device includes an inflation sealing cuff.

31. The kit of claim 30, wherein the kit includes instructions to deliver the viscoelastic material to an area adjacent the inflation sealing cuff of the medical device within the body cavity such that the viscoelastic material provides enhanced impermeability to the inflation sealing cuff of the medical device.

32. The kit of claim 29, wherein a delivery means is provided to deliver the viscoelastic material into the body cavity.

33. The kit of claim 32, wherein the delivery means is a syringe, needle, cannula, catheter, tubing, or pressure applicator.
